(19) United States
(12) Patent Application Publication
(10) Pub. No.: US 2019/0366374 A1
(43) Pub. Date: Dec. 5, 2019

(54) SMART SHOWER HEAD

(71) Applicant: Intellihot, Inc., Galesburg, IL (US)

(72) Inventors: Sridhar Deivasigamani, Peoria, IL (US); Sivaprasad Akasam, Dunlap, IL (US)

(21) Appl. No.: 16/540,124

(22) Filed: Aug. 14, 2019

Related U.S. Application Data
(63) Continuation-in-part of application No. 15/239,777, filed on Aug. 17, 2016.
(60) Provisional application No. 62/205,772, filed on Aug. 17, 2015.

(51) Int. Cl. B05B 12/10; B05B 12/12; B05B 12/00; G05B 13/02

(52) U.S. Cl. CPC B05B 12/10; B05B 12/122; B05B 1/18; G05B 13/0265; B05B 12/004

(57) ABSTRACT

A shower head adapted to receive a supply of water from a water heater, the shower head including: a controller functionally connected to the water heater; and a height sensor functionally coupled to the controller, wherein the height sensor is configured to output a signal corresponding to the height of a user within a detection range of the height sensor, the height of the user is configured to correspond to a pre-determined setpoint temperature of the user, wherein the controller is configured for communicating the pre-determined setpoint temperature to the water heater for controlling the supply of water to the pre-determined setpoint temperature.

### PRIORITY CLAIM AND RELATED APPLICATIONS

This continuation-in-part application claims the benefit of priority from non-provisional application U.S. Ser. No. 15/239,777 filed Aug. 17, 2016, which in turn claims priority from provisional application U.S. Ser. No. 62/205,772 filed Aug. 17, 2015. Each of said applications is incorporated by reference in its entirety.

### BACKGROUND OF THE INVENTION

### 1. The Field of the Invention

The present invention is directed generally to a shower head. More specifically, the present invention is directed to a smart shower head capable of aiding in providing hot water at various setpoint temperatures to a user at opportune times to make showers more comfortable and reduce wastes in water and energy.

### 2. Background Art

Delays in obtaining shower water at a suitable temperature has been a source of discontent for many users. Even with modern tank or tankless heat exchangers having both external and internal recirculation systems, there can still be a short portion of the shower water supply line at or near the shower head that contains unheated water. For those stepping in the shower before or immediately after it is turned on, the user is almost ensured cold water especially if the shower has not been used for an amount of time it takes the water trapped between the water heater and the shower head to cool down. Further, the control of water temperature is largely an activity that is learned over time for the particular shower and that is re-established every time a user is to use the shower. For instance, upon entering a shower and upon experiencing the shower water temperature, the user may adjust the shower handle setting for a desired mixing ratio of the hot and cold water supplies to achieve a desired shower water temperature. Further, upon getting accustomed to an initial water temperature, the user may then desire a different, higher water temperature to feel comfortable. Despite concerns of water, time and energy wastage, many users are still taking long showers especially if the cost of doing so is covered by an umbrella fee that has already been paid, e.g., in a hotel, etc., and that the additional costs incurred to the users are small or negligible compared to the pleasure derived from taking long showers. Therefore, there lacks incentives in conserving water, time and energy while the user is taking shower. Further, when a user is taking a long shower, the user is not otherwise occupied by any other activities. Opportunities are lost if the user is already in a position to perform important daily or routine tasks, e.g., taking vital signs, etc., but does not take advantage of such opportunities. Yet further, there lacks a simple means for programming a water heating system to perform water temperature acclimation adjustment, e.g., automatic adjustment including increasing the setpoint temperature based on usage duration. Yet further, there lacks a means or a simple means for customizing services of a shower. For instance, every member of a family may have a preference for the shower setpoint temperature and the rate at which this setpoint temperature is increased. If a user is required to take several steps to self-identify to customize every shower service, the user may be dissuaded to do so. However, if the user simply needs to customize his shower services once before use, he would be more keen to utilize the services.

At least one prior attempt has been made to encourage users of a shower to reduce unnecessary shower time as disclosed in U.S. Pat. No. 7,948,831 to Orcutt et al. (hereinafter Orcutt) which discloses a sound device with a motion detector. The device is used to provide a diversion while user occupies the shower, at the same time keeping track of time and alarming the occupant when time has elapsed. For example, a minute after entering the shower, the timer automatically starts and a trivia question is posed to the occupant. The occupant then has a predefined amount of time to come up with the answer. Upon time lapse, the answer is provided with a gentle reminder that the user's time is over. However, Orcutt does not disclose services geared towards making showers more comfortable with setpoint temperature adjustments, etc.

Thus, there is a need for a smart shower head capable of being programmed to perform a number of tasks, e.g., automatic setpoint temperature adjustment and to perform the tasks according to user preferences. There is also a need for a device to automatically identify a user, thereby removing the need for the user to self-identify per use to effectuate a setpoint temperature or other settings.

### SUMMARY OF THE INVENTION

In accordance with the present invention, there is provided shower head adapted to receive a supply of water from a water heater, the shower head including: a controller functionally connected to the water heater; and a height sensor functionally coupled to the controller, wherein the height sensor is configured to output a signal corresponding to the height of a user within a detection range of the height sensor, the height of the user is configured to correspond to a pre-determined setpoint temperature of the user, wherein the controller is configured for communicating the pre-determined setpoint temperature to the water heater for controlling the supply of water to the pre-determined setpoint temperature.

In one embodiment, the controller is configured to automatically change the pre-determined setpoint temperature upon a duration of use of the shower head to increase the comfort of the user. In one embodiment, the controller is configured to automatically increase the pre-determined setpoint temperature upon a duration of use of the shower head to increase the comfort of the user.

An object of the present invention is to provide a shower head capable of automatic adjustment of a shower setpoint temperature.

Another object of the present invention is to provide a shower head capable of identifying the user of the shower head.

Another object of the present invention is to provide a shower head capable of automatic adjustment of shower setpoint temperature based on the identity of the user.

Another object of the present invention is to provide a shower head capable of providing additional services that a user of the shower head may receive while using the shower head.

Whereas there may be many embodiments of the present invention, each embodiment may meet one or more of the foregoing recited objects in any combination. It is not intended that each embodiment will necessarily meet each objective. Thus, having broadly outlined the more important features of the present invention in order that the detailed description thereof may be better understood, and that the present contribution to the art may be better appreciated, there are, of course, additional features of the present invention that will be described herein and will form a part of the subject matter of this specification.

### BRIEF DESCRIPTION OF THE DRAWINGS

In order that the manner in which the above-recited and other advantages and objects of the invention are obtained, a more particular description of the invention briefly described above will be rendered by reference to specific embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings in which:

FIG. 1 is a diagram depicting various components functionally connected to a controller according to one embodiment of the present shower head.

FIG. 2 is a diagram depicting one embodiment of the present shower head, wherein no flow is allowed through a diverter and a shower head.

FIG. 3 is a diagram depicting one embodiment of the present shower head, wherein a flow is allowed through a diverter but no flow is allowed through a shower head.

FIG. 4 is a diagram depicting one embodiment of the present shower head, wherein a flow is allowed through a shower head but no flow is allowed through a diverter.

FIG. 5 is a diagram depicting one embodiment of the present shower head, wherein a height sensor is useful for initiating the adjustment of the setpoint temperature of the water heater supplying water to the shower head.

FIG. 6 is a general block diagram depicting basic signal logic relationships among the controller, the physical attribute sensor(s) and the water heater.

FIG. 7 illustrates a height-detecting zone chart showing an example of a default user detection scheme for a shower head.

FIG. 8 illustrates an exemplary graph containing time-based height signatures for various triggering entities (toddler, child and adult).

FIG. 9 illustrates a height-detecting zone chart.

### PARTS LIST

2—shower head
4—controller
6—onboard valve
8—hydrogenerator
10—height sensor
12—demand sensor, e.g., proximity sensor
14—temperature sensor
16—communication device
18—battery
20—fluid conductor
22—direction in which fluid flows
24—heart rate monitor
26—touch sensor
28—blood pressure monitor
30—water heater
32—flow sensor
34—diverter
36—valve block
38—bypass path
40—valve spool
42—solenoid driver
44—user
46—water heater controller
48—setpoint temperature
50—interface
52—signal receiver
54—step of controlling water heater to new setpoint temperature
56—shortest distance between height sensor and user

### PARTICULAR ADVANTAGES OF THE INVENTION

In one embodiment, the present smart shower head enables customization of water heater setpoint temperature based on users' height. Upon initial setup for each user, no further identification equipment, code or setup routine is required of the users. Previously, the setpoint temperature of a water heater is not customizable from a shower head. The present shower head provides not only a means for customizing the water heater setpoint temperature, but also a means for customizing the water heater setpoint temperature for multiple users. The selection of a setpoint temperature is made automatically and on-the-fly based on a user's height.

In one embodiment, the present smart shower head requires no additional disparate input interfaces to receive user input. Instead, all of the required input interfaces are made available on the shower head, making the shower head a comprehensive tool which enables initial setup for customization and subsequent adjustment of the water heater setpoint temperature, all within one unit.

The availability of a temperature sensor and a valve in the present smart shower head allows the detection of water of unsuitable temperature and a means to avoid transmitting the water to the user. In contrast, the use of a temperature sensor disposed at a water heater located at a great distance from the shower head does not provide an accurate indication of the temperature of the water that a user will receive when a shower commences. Further, there is a distinct point where the temperature of the water supplied through the shower head has been suitably prepared, i.e., the desired water temperature has been reached at the shower head.

In one embodiment, a diverter is provided to purge the trapped water that is disposed at a temperature not meeting the setpoint temperature setting of the water heater, in a direction away from a user, thereby preventing water of unsuitable temperature from contacting an unsuspecting user.

Privacy concerns preclude the use of imaging-type sensors, cameras or other imaging equipment for user identification, e.g., via facial recognition, etc., especially when users are taking a shower, about to take a shower or otherwise in a vulnerable situation to be embarrassed if access to images obtained through imaging-type sensors, cameras or other imaging equipment is compromised. A height sensor is used herein instead, addressing any privacy concerns as the height sensor lacks the capability of recording images and therefore lacks the risk of being tampered for access to images. A shower head provides a base upon which the height sensor can be readily mounted and as the shower head is a point of use of a user (i.e., the user must position himself or herself under the shower to take a shower), the sensor data obtained via the height sensor can be reliably used to identify the user based on the user's height.

### DETAILED DESCRIPTION OF A PREFERRED EMBODIMENT

The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

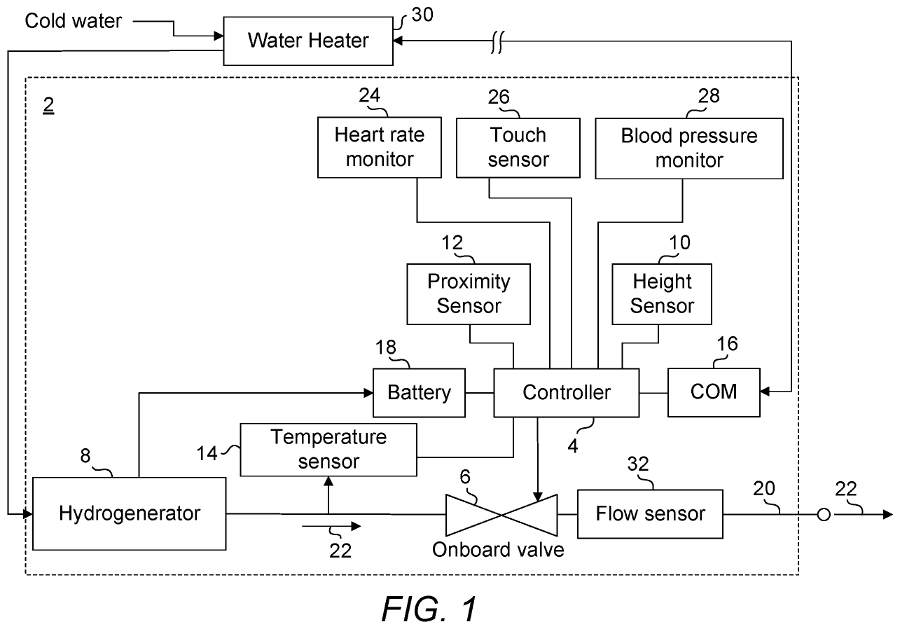

FIG. 1 is a diagram depicting various components functionally connected to a controller according to one embodiment of the present shower head. Disclosed herein is a shower head 2 adapted to receive a supply of water from a water heater. The shower head 2 includes a controller 4 functionally connected to the water heater 30, a demand sensor, e.g., proximity sensor 12 for detecting a hot shower demand, wherein the proximity sensor 12 is functionally coupled to the controller 4. In one embodiment, a hot shower demand is indicated by the detection of an object within the detection range of the proximity sensor 12. There is further disclosed a temperature sensor 14 functionally coupled to the controller 4 and adapted to measure the temperature of the supply of water at the shower head; and a valve 6 functionally coupled to the controller 4. In one embodiment, the valve controls whether the supply of water is allowed to exit via the shower head, a diverter or it shall be terminated. In one embodiment, if a hot shower demand is determined by the controller 4 to exist and the temperature of the supply of water is determined by the controller 4 to be outside of a range of ideal temperature, the valve 6 is opened to allow draining of the supply of water until the temperature of the supply of water falls within a range of ideal temperature and if the hot shower demand continues to exist, the valve is configured to remain open. In one embodiment, an ideal temperature is about the setpoint temperature of the water heater.

In one embodiment, the shower head further includes a physical attribute, e.g., height sensor 10 functionally coupled to the controller 4, wherein the height sensor 10 is configured to detect the height of a user within the detection range of the height sensor, determining the setpoint temperature of the water heater based on the height of the user, and therefore the user's identity and the user's setpoint temperature setting and communicating the setpoint temperature to the water heater 30.

In one embodiment, the shower head 2 further includes a communication device 16 functionally connected to the controller 4. In one embodiment, the communication device is a wireless communication device capable of Wireless Fidelity (Wi-Fi) communication. In one embodiment, the communication device is a wireless communication device capable of Bluetooth® communication. Other means of communication are possible but preferably via a ubiquitous wireless protocol that is widely available. The function of the communication device 16 is three-fold. It is configured to: (i) receive commands and/or data actionable within the shower head itself; (ii) transmit commands and/or data to a target device, e.g., a water heater or another device, where the commands and/or data are actionable within the target device; and (iii) receive commands and/or data from a first device and passing the received commands and/or data to a second device. In one embodiment, the setpoint temperature of the water heater is configured to be adjustable remotely. In one embodiment, the setpoint temperature of the water heater is determined based on the shower mode selected at the shower head.

In one embodiment, the shower head 2 further includes an on-board hydrogenerator 8 adapted to receive the supply of water and generate power from the supply of water to power the shower head. Suitable hydrogenerators 8 include, but not limited to, a hydrogenerator disclosed in U.S. Pat. No. 7,233,078 to Baarman et al. and U.S. Pat. No. 7,253,536 to Fujimoto et al. Electricity generated from the hydrogenerator 8 is then stored in a rechargeable battery 18 for use by the controller 4, sensors and monitors 10, 12, 14, 24, 26, 32, communication device 16, valve 6 and any other devices requiring power to operate.

In one embodiment, the shower head “learns” the shower usage pattern of a user by recording shower usage over time and determining time periods where the frequency of the shower usage are the highest and build a pattern of water usage. Hot water can then be prepared at such time periods to reduce delays associated with preparing hot water. Signals indicating the starting time and ending time of a shower and flowrates (as collected at flow sensor 32) can be communicated to a water heater controller such that the data may be collected and used according a hot water preparation routine, where the control of hot water preparation is given to the water heater controller. Alternatively, the data may be stored in an onboard memory of the shower head controller 4. Hot water may be prepared according to a control mechanism involving historical flow demand as disclosed in U.S. Pat. Pub. No. 20140229022.

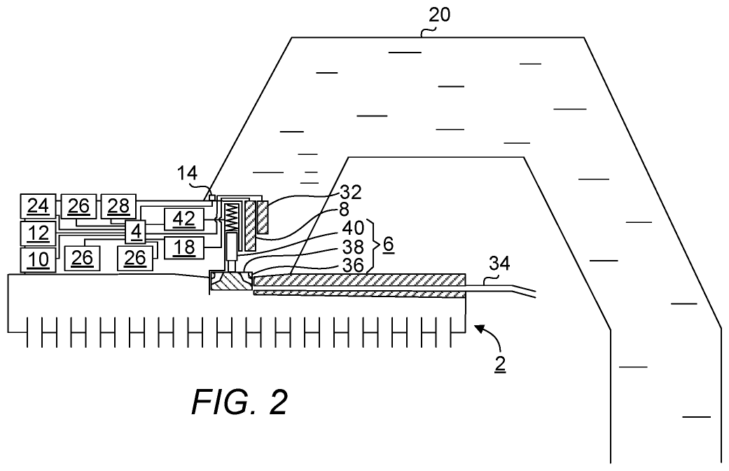

In one embodiment, the shower head is equipped with a device such that the direction in which it points can be altered. In this embodiment, if hot water cannot be provided without first draining a reserve of cold water, the shower head is configured to point away from the user, e.g., toward a shower wall before the valve is opened to drain the cold water. This way, the user can avoid getting a cold shower when hot water is desired. When the shower head eventually receives suitably hot water as indicated by the temperature sensor 14, the shower head 2 is allowed to supply hot water in a direction towards the user. In order to avoid dispensing cold water on a user, upon detecting a desire to use hot water, the shower head is configured to empty the reserve of cold water at the shower head by opening its valve until such time when the shower detects a flow already disposed at a suitable temperature. FIGS. 2-4 depict one example where a supply of water from a water heater is selectively dispensed at a shower head. FIG. 2 is a diagram depicting one embodiment of the present shower head, wherein its valve is disposed in a position allowing no flow through a diverter and a shower head. It shall be noted that no flow has been established through a diverter 34 or the shower head 2. In the embodiment shown, the valve 6 is essentially a three-position spring-return solenoid valve having a valve block 36-equipped spool 40 where the position of the valve block 36 dictates whether a flow is halted, supplied through bypass path 38 and the diverter 34 or supplied through the shower head while the diverter 34 is blocked. Several other positions are also possible for providing several shower patterns, e.g., for pre-wash, soaping, rinse, oscillating flow and pressure (muscle relaxant), etc.

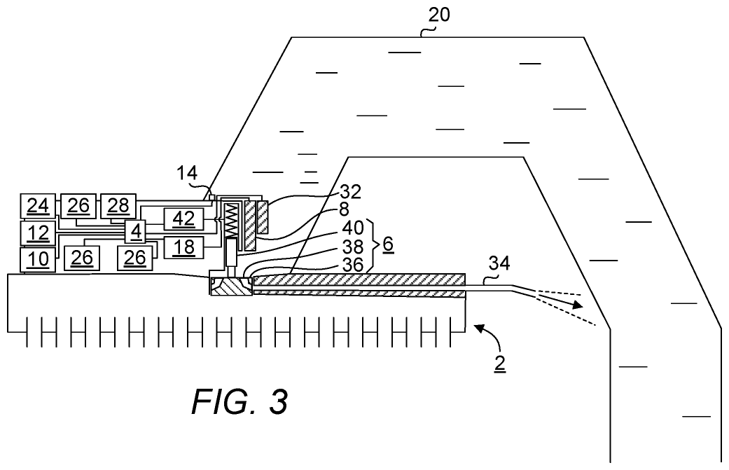

FIG. 3 is a diagram depicting one embodiment of the present shower head, wherein a flow is allowed through a diverter but no flow is allowed through a shower head. It shall be noted that no flow is allowed through the shower head but the valve 6 is disposed in a position such that the diverter 34 allows a flow to occur to empty the water trapped within the shower head. It shall also be noted that the opening of the diverter 34 points a flow through it in a direction that is not aimed downwardly to avoid purging an unsuitably heated supply of trapped water onto the user. Upon detecting a hot water demand and if the temperature sensor 14 detects a temperature that is unsuitable for the user's consumption, e.g., if the water temperature, as detected by the temperature sensor 14, as being too low, e.g., more than 2 degrees F. lower than the setpoint temperature of water heater, the water trapped in the shower head will be purged via diverter 34 until such time when the temperature sensor 14 registers a temperature that falls within a range considered suitable for the user's consumption, e.g., 2 degrees F. above or below the setpoint temperature of the water heater.

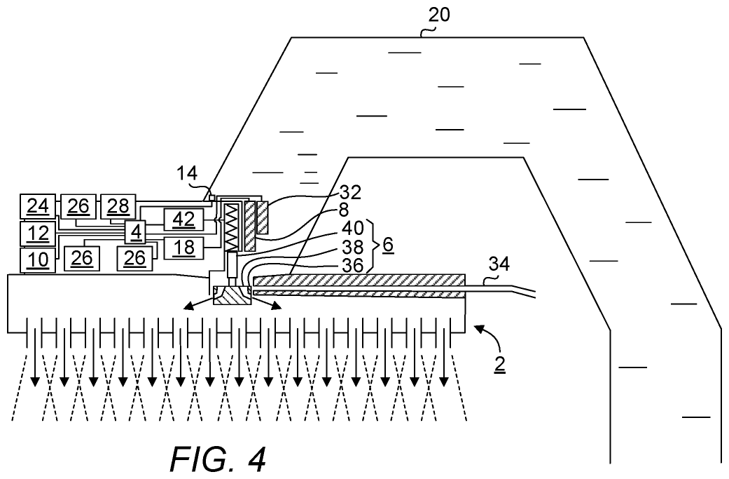

FIG. 4 is a diagram depicting one embodiment of the present shower head, wherein a flow is allowed through the shower head but no flow is allowed through the diverter 34.

When the water temperature, as detected at temperature sensor 14, is considered suitable for a user's consumption, the spool 40 is driven to a position where a flow can occur through the bypass flow path 38 of the valve block 36 into the shower head and subsequently onto a user.

In one embodiment, a desire to use hot water is indicated by a persistent presence of an object in the detection view of a presence sensor. In one example, a persistent presence is defined as a presence that lasts for more than two seconds. In another example, a persistent presence is defined as a presence that lasts for more than four seconds. In another embodiment, a desire to use hot water is indicated by a presence of an object followed by an absence of the object in the detection view of a presence sensor. This pattern can be a wave of the hand of a user. Upon detecting a wave of a hand, the shower starts to run to empty the cold water still trapped in a portion of the shower line at or near the shower head that contains unheated water. The shower then stops and waits for the user to get into the shower, preventing hot water wastage. In this embodiment, no diverter is necessary as a user does not place himself or herself under the shower head and there is little chance that the user will experience the trapped water with unsuitable temperature.

It is possible that a user may enter and stay in a shower without first waving his hand at the shower head. In one embodiment, if hot water cannot be provided without first draining a reserve of cold water, the valve is configured to not open until the user stepped away from the shower.

In one embodiment, a desired shower temperature is selected, e.g., 105 degrees F. A wave of a hand in the shower indicates that the desired water temperature is desired and not the default temperature, e.g., setpoint temperature of the water heater of 120 degrees F. A valve disposed at the shower head then allows the shower head to be emptied while water flow of the desired temperature is being prepared at the water heater. When water flow of the desired temperature reaches the shower head, the valve is closed to stop the water flow and anticipate water usage by the user. In one embodiment, the water temperature of a shower is tailored to an individual's preference. The shower head 2 is configured such that it communicates the desired water setpoint temperature that is based on a user's preference to the water heater 30. In one embodiment, the user is identified by his or her height. The shower head 2 communicates a base temperature set at a predetermined level that is lower than the temperature needed by any user potentially using the shower to the water heater 30 responsible for supplying hot water to the shower head. For instance, the base may be set at 100 degrees F. Upon identifying the individual, the water heater setpoint temperature is then altered to a level suitable for the individual. In order to provide a user-specific setpoint temperature at the water heater, the shower head must first be taught. For instance, if a user of a particular height takes a shower at a particular setpoint temperature previously, such setpoint temperature is recorded in a database, e.g., stored in a memory, of the smart shower head 2 or the water heater 30. If a user of this height is again detected, the previously set setpoint temperature for the user can be used again. It is also possible that as the shower head 2 has a means for associating a setpoint temperature to a user of a particular height and a means for detecting the water temperature, a user may physically turn the water supply to any setting (e.g., any ratio of hot and cold water supplies) and still receive water of temperature previously used and learned by the shower head 2. In order to reduce the chance that this is not the preferred water temperature setting by the user, this setting is only activated when the user is detected at the particular time of day, i.e., this setting is most useful for users adhering to a daily routine. In yet another embodiment, a shower head 2 can vary the setpoint temperature automatically with respect to the duration from which the shower head was first turned on. For instance, for every minute that the shower head 2 has been allowing flow, the water heater setpoint temperature can be increased by 1 degree F. When a user first enters a shower, the user may be initially satisfied with a water temperature that is higher than the ambient air temperature. The user will likely demand progressively hotter water as the shower progresses due to user temperature acclimation. Normally, a user demands hotter water by manually adjusting a valve adapted for adjustment of the flowrate ratio of the heated and unheated water with a handle. The handle provides grasp points for controlling the amount of hot water supply that is mixed with the cold water supply. The present shower head 2 optionally includes a feature that allows the user to set an automatic temperature rise rate and a high temperature limit that aligns with the user's temperature rate of change profile or acclimation profile; thereby providing a varying temperature service. For example, the initial requested temperature is 90 degrees F. The user may choose to increase the water temperature by 1 degree per minute. If the demand is left untouched, the water temperature would be increased to 95 degrees in 5 minutes of usage. If the high temperature limit is set at 92 degrees, the water temperature would be increased only to 92 degrees in 2 minutes and remains at that temperature throughout the rest of the duration of the demand. In another example, a user may prefer to take shower that gets progressively cooler. In this case, the present shower head 2 includes a feature that allows the user to set an automatic temperature drop rate and a low temperature limit that aligns with the user's temperature rate of change profile or acclimation profile; thereby also providing a varying temperature service.

When a user is finished with his shower and exits the shower, the shower head detects a change in state as indicated in the user presence sensor and automatically stops the shower and prevents waste. A delay may be built in to ensure that the user has truly exited the shower and is not simply moving about in the shower to possibly cause a false interpretation by the presence sensor 12 that the user has exited the shower. In one embodiment, the frequency of detected “touches” of “contacts” made by a user is used to determine the user intent. In one embodiment, an input interface, e.g., a touch sensor is provided. In this embodiment, the intent to increase the shower setpoint temperature is signified by a motion which generates at least three taps of touches at the touch sensor 26 of the shower head 2 at similar frequencies. For instance, if three touches are detected where they are spaced at periods of about 1 second, the three touches may be considered an intent to increase the temperature of the water supply, and thus causing the shower controller 4 to communicate such intent to the water heater controller. However, if the periods of the three touches are about 0.5 second, the three touches may be considered an intent to decrease the temperature of the water supply, and thus causing the controller to communicate the intent to lower the temperature of the water supply to the water heater controller. The intent to lower the temperature of the water supply may be indicated by actions that take a smaller amount of time as this intent shall be met with an action that is more urgent, e.g., due to intolerably hot water as compared to the action to increase the temperature.

In one embodiment, the shower head 2 further includes a heart rate monitor 24. An example of a suitable heart rate monitor includes, but not limited to, an optical heart rate monitoring (OHRM) device. In one embodiment, the shower head 2 further includes a blood pressure monitor 28. Examples of suitable blood pressure monitors include, but not limited to those cuff-less monitors available through Viatom® and Maisense®. Each of these devices is incorporated in the shower head 2. Therefore, in using these devices, one simply needs to place one or two fingers upon the sensing, receiving or interfacing surfaces of these devices 24, 28. In one embodiment, such contact is automatically detected and that a routine for measuring the user's heart rate or blood pressure is automatically started and completed while the one or two fingers are placed on the sensing surfaces. In another embodiment, a switch is provided, where upon being triggered, the routine for measuring some aspects of the user's vital signs are started and run to completion. In one embodiment, the data obtained from vital signs measurements is transmitted to a central server via communication device 16 where the data can then be retrieved on a computer or a smart phone. In one embodiment, the shower head controller further records diagnostic data, e.g., time, water flowrate, water temperature, etc., over time to a memory functionally connected to the shower head controller or the water heater controller responsible for preparing water for the shower head 2 or a central server available remotely from the shower head 2.

In yet another embodiment, an imminent need for hot water is indicated by another demand sensor. In this example, the imminent need is indicated by the movement of a toothbrush or the flushing of a toilet. The use or movement of a toothbrush or the flushing of a toilet may be indicated by any one of the following activities. In one embodiment, the toothbrush and/or its cradle (configured for holding or supporting the toothbrush) or other peripheral devices is equipped with a switch capable of indicating whether or not the toothbrush has been removed from its cradle. In another embodiment, the removal of a Radio Frequency Identification (RFID)-tagged toothbrush from the detection range of its RFID-equipped receiver indicates that the toothbrush is in use and the user may imminently require hot water services. In one embodiment, a switch configured to detect the flushing action of a toilet is provided. Such indication of removal of toothbrush or flushing of a toilet may then be communicated via a communication protocol, e.g., Wi-Fi, Bluetooth, etc., to the communication device 16 of controller 4 such that the need for hot water can be communicated to a corresponding water heater and hot water can start being prepared in anticipation of an imminent use. Alternatively or additionally, such indication may also be communicated via a communication protocol to the controller of a water heater. It is also possible to distinguish whether an imminent use is likely by determining if the use of a toothbrush occurs in the morning or at night. For instance, it may be more likely for one to continue on to take a shower after brushing teeth in the morning than at night. Therefore, hot water preparation may be turned on in anticipation of a shower only or in anticipation of both a shower and teeth-brushing. In another embodiment, both shower head water preparation, e.g., emptying of trapped unsuitably heated water in the shower head and hot water preparation at the water heater may be made simultaneously while a demand has been detected by any one of the demand sensors disclosed elsewhere herein.

In yet another embodiment, the toothbrush can also have a heart rate monitor and a blood pressure monitor. Therefore, heart rate and blood pressure of a user may be taken while the user is brushing teeth, negating the need for the user to take such measurements separately.

In yet another embodiment, the setpoint temperature of a water heater functionally connected to a present shower head is adjusted according to the flow pattern of the shower head. For instance, if a shower pattern of misting is used, the shower may come across as colder to a user of the shower. In one embodiment, the valve position which corresponds to a flow pattern is communicated to the water heater so that the setpoint temperature of the water heater can be increased to increase user comfort at the shower. In one embodiment, a setpoint temperature of the water heater is determined in the shower head controller based on the valve position which corresponds to a flow pattern. The setpoint temperature is then communicated to the water heater such that the temperature of the shower flow can be increased to increase user comfort at the shower.

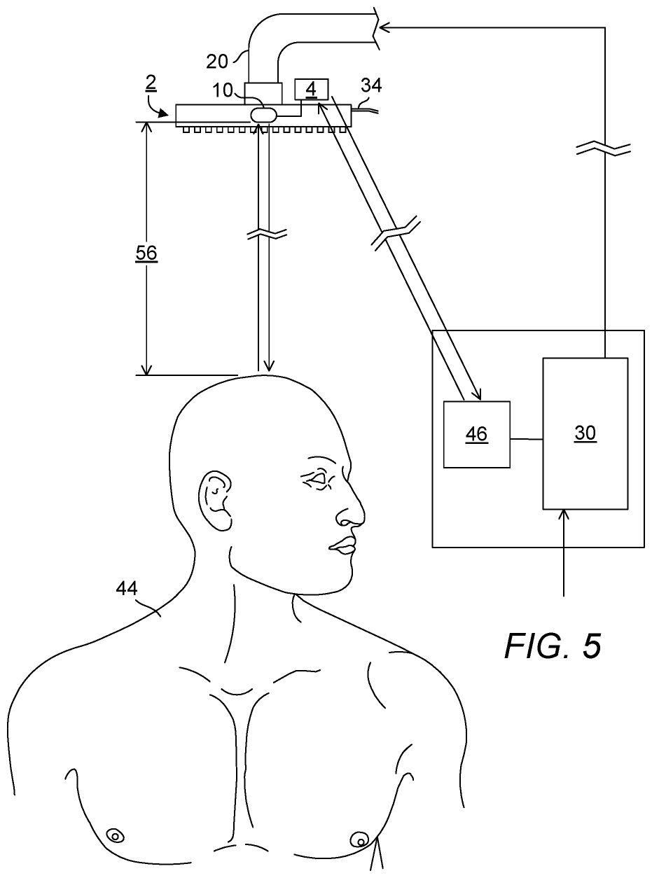

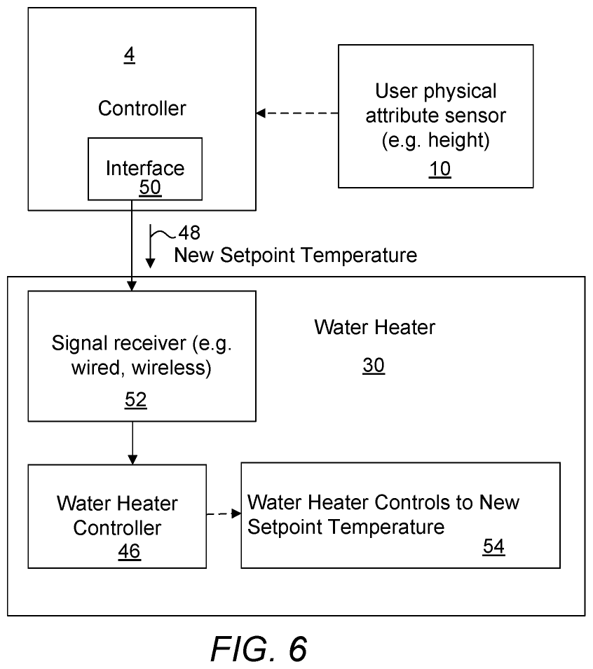

FIG. 5 is a diagram depicting one embodiment of the present shower head, wherein a height sensor 10 is useful for initiating the adjustment of the setpoint temperature of the water heater supplying water to the shower head. The height of a user 44 is derived from the shortest distance 56 between height sensor 10 and the user 44, i.e., the distance between the height sensor 10 and the top of the head of the user, and the distance between the height sensor 10 and the shower floor upon which the user 44 is supported. The distance between the height sensor 10 and the floor is established after installation of the shower head when the shower of the shower head is cleared of a user. The height of a user is the difference between the two distances. FIG. 6 is a general block diagram depicting basic signal logic relationships among the controller, the height sensor 10 and the water heater 30. In one embodiment, once a physical attribute, e.g., height, signature is identified and confirmed by controller 4, the controller 4 sends a new setpoint temperature to signal receiver 52 that functions as a signal interface for water heater 30 regardless of whether the setpoint temperature controlled to by the water heater 30 differs from the new setpoint temperature. A new setpoint temperature 48 can be transmitted using a hard wired connection as well via a wireless means. In one embodiment, a determination is made whether or not to send a new setpoint temperature to the water heater controller 46. If the setpoint temperature corresponding to the user 44 is determined to be different from the value held by the water heater controller 46 and controlled to as in step 54 by the water heater 30, a new setpoint temperature is issued as shown in FIG. 6, otherwise no new setpoint temperature will be communicated to the water heater controller 46. Such determination may also be made by the water heater controller 46 instead of the shower head controller 4. If the latter strategy is chosen, a setpoint temperature is sent from controller 4 to the water heater controller 46 regardless of whether the water heater is already controlled to supply water at the setpoint temperature associated with the user.

The typical steps a shower head would go through in adjusting the water setpoint temperature begins with detecting a user in the shower and generating a physical attribute, e.g., height, signature corresponding to the user. The step is then followed by comparing and selecting the user's generated physical attribute signature to a user signature database, and selecting a best match user signature that best aligns with the user's physical attributes. At this point, the system retrieves a setpoint temperature corresponding to the best match or closest user signature. Finally, the last step involves activating the setpoint temperature for the water heater, i.e., controlling the water heater to output water at the setpoint temperature.

Physical attribute sensor(s) is comprised of at least one sensor capable of detecting and measuring at least one physical attribute, e.g., the height, of a hot water user. The use of more than one sensor has advantages, e.g. reduction in false triggering. Available sensors include: height or distance sensors, pressure (weight) sensors, light or laser based sensors, ultrasonic sensors, or any combination thereof. In preferred embodiments, a sensing system will provide a reliable, safe, non-obtrusive, hardware and associated methods of detection. Additionally, relatively inexpensive, easily installed sensing systems are considered desirable attributes of preferred embodiments. Most of the aforementioned sensing systems can be designed to decipher motion as well as distance via the analysis of the parameter being detected. One such preferred sensor is the ultrasonic based sensing system. The following is an excerpt from a published lecture available from Brown University of Providence, R.I., reviewing the fundamentals of ultrasonic sensing.

### Ultrasonic Acoustic Sensing

Ultrasonic sensors are often used in robots for obstacle avoidance, navigation and map building. Much of the early work was based on a device developed by Polaroid for camera range finding. From the Hitechnic Ultrasonic Sensor web page we learn that their “ultrasonic range sensor works by emitting a short burst of 40 kHz ultrasonic sound from a piezoelectric transducer. A small amount of sound energy is reflected by objects in front of the device and returned to the detector, another piezoelectric transducer. The receiver amplifier sends these reflected signals (echoes) to [a] micro-controller which times them to determine how far away the objects are, by using the speed of sound in air. The calculated range is then converted to a constant current signal and sent to the RCX.” The Hitechnic sensor is different from the Polaroid sensor in that it has separate transmitter and receiver components while the Polaroid sensor combines both in a single piezoelectric transceiver; however, the basic operation is the same in both devices.

There are a number of complications involved in interpreting the time-of-flight information returned by an ultrasonic sensor. If the sensor face is parallel to the surface of the nearest object and that surface is flat, reflective and relatively large, e.g., a plaster wall, then the information returned by the sensor can be reasonably interpreted as the distance to the nearest object in front of the sensor. However if the object deviates significantly from this ideal object, the time-of-flight information can be misleading. Here is one of the more benign sorts of interpretation error caused by the fact that the signal (corresponding to a propagating wave of acoustic energy) spreads as it propagates further from the sensor with most of the energy of the leading edge confined to a 30-degree cone. If the surface is angled with respect to the face of the sensor (as it is below) then the time of flight information will record the distance to nearest point within the 30-degree cone. (End of quote)

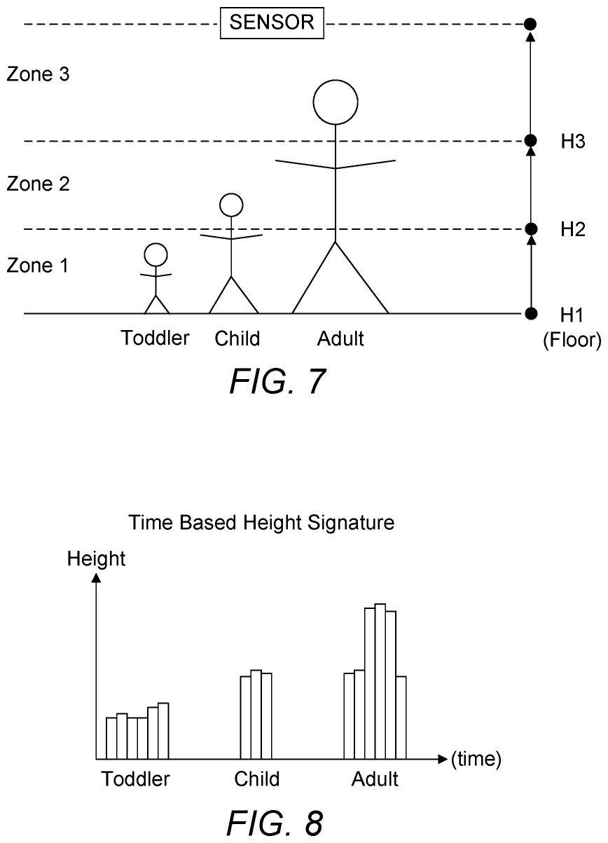

Referring again to FIG. 6, the exemplary ultrasonic sensor-based sensing system is clearly able to decipher distance or height via the analysis of the acoustic transmissions and subsequent reflections through air. Such a system provides a time-based height signature that is able to detect scanned entities or hot water users that possess different physical attributes, e.g., height, as depicted in FIGS. 7 and 8.

FIG. 7 illustrates a height-detecting zone chart showing an example of a default user detection scheme for a shower head. FIG. 8 illustrates an exemplary graph containing time-based height signatures for various triggering entities (toddler, child and adult). Controller 4 is configured to detect various types of hot water users either as a unique individual user, a general category user, or any combination thereof. Exemplary entities, depicted in FIG. 7 include a toddler, a child and an adult and their corresponding respective time-based height signatures are depicted in FIG. 8.

Controller 4, controller interface 50, and physical attribute sensor(s) 10 cooperate such that detected entity or a user are properly classified via a physical attribute signature. Again, the methods and associated hardware for detecting and comparing sensor signals, along with activating signal controllable mechanisms such as blowers, burners, and valves is a well-known, mature water heater technology and implementation would not present an undue burden to those versed in the art.

Referring to FIG. 7, the chart depicts an exemplary signature detection scheme where three height zones or general category provide the basis for user categorization. The right and left vertical axes are defined as follows:

Zone 1: The height detection range that includes all entities whose detected height is below height value H2 and greater than or equal to height value H1 (floor level). Exemplary entities included in this zone include: toddlers and the like.

Zone 2: The height detection range that includes all entities whose detected height is below height value H3 and greater than or equal to height value H2. Exemplary entities included in this zone include: children and the like.

Zone 3: The height detection range that includes all entities whose detected height is greater than or equal to height value H3. Exemplary entities included in this zone include: adults.

Exemplary system responses resulting from various entity signatures based on the time-based height signatures are depicted in FIG. 8.

A toddler is categorized as a zone 1 entity. The corresponding signal is classified as a heat sensitive user and water heater controller 46 controls the water heater 30 to a setpoint temperature configured for a toddler hot water user. Maximum temperature will be automatically set to a predetermined toddler safe value (e.g., 100 degrees F.) to prevent burns.

A child is categorized as a zone 2 entity. The corresponding signal is classified as a heat sensitive user and water heater controller 46 controls the water heater 30 to a setpoint temperature configured for a child hot water user. Maximum temperature will be automatically set to a predetermined child safe value (e.g., 105 degrees F.) to prevent burns although a child is now more tolerant than a toddler to hot water.

An adult is categorized as a zone 3 entity. The corresponding signal is classified as an adult user and water heater controller 46 controls the water heater 30 to a setpoint temperature configured for an adult hot water user. Maximum temperature will be automatically set to a predetermined adult value (e.g., 120 degrees F.) to align with adult expectations.

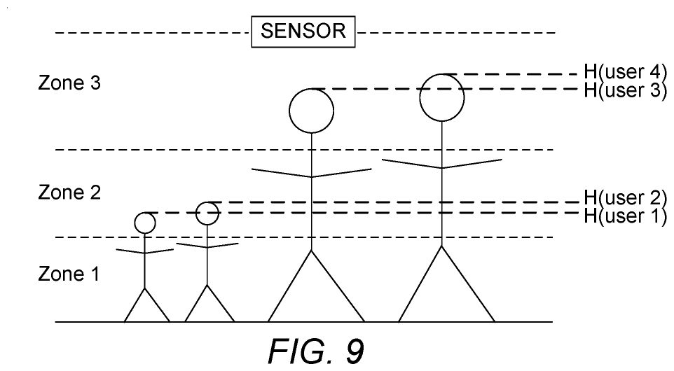

FIG. 9 illustrates a height-detecting zone chart. Depicted is an identification scheme that detects a unique individual user via time-based height signature. The depiction differs from that of FIG. 7 in that classification scheme of FIG. 7 is directed to placement of the detected user into general category. Unique individual users are labeled as H(user 1) to H(user 4). In an exemplary set up, water heater 30 can provide a personalized maximum water temperature T(maximum) response output for each specific user. A learning step or procedure to input the unique individual preferences such as height signature and the maximum setpoint temperature into controller 4 would be accomplished via interface 50, or the like. A system is capable of such personalized recognition can be used to align special hot water requirements or preferences to accommodate special circumstances; e.g. an elderly adult concerned with possible burns, due to feeling loss, can possess a lower maximum setpoint temperature setting.

The detailed description refers to the accompanying drawings that show, by way of illustration, specific aspects and embodiments in which the present disclosed embodiments may be practiced. These embodiments are described in sufficient detail to enable those skilled in the art to practice aspects of the present invention. Other embodiments may be utilized, and changes may be made without departing from the scope of the disclosed embodiments. The various embodiments can be combined with one or more other embodiments to form new embodiments. The detailed description is, therefore, not to be taken in a limiting sense, and the scope of the present invention is defined only by the appended claims, with the full scope of equivalents to which they may be entitled. It will be appreciated by those of ordinary skill in the art that any arrangement that is calculated to achieve the same purpose may be substituted for the specific embodiments shown. This application is intended to cover any adaptations or variations of embodiments of the present invention. It is to be understood that the above description is intended to be illustrative, and not restrictive, and that the phraseology or terminology employed herein is for the purpose of description and not of limitation. Combinations of the above embodiments and other embodiments will be apparent to those of skill in the art upon studying the above description. The scope of the present disclosed embodiments includes any other applications in which embodiments of the above structures and fabrication methods are used. The scope of the embodiments should be determined with reference to the appended claims, along with the full scope of equivalents to which such claims are entitled.

What is claimed herein is:

1. A shower head adapted to receive a supply of water from a water heater, said shower head comprising:
(a) a controller functionally connected to the water heater; and
(b) a height sensor functionally coupled to said controller, wherein said height sensor is configured to output a signal corresponding to the height of a user within a detection range of said height sensor, the height of the user is configured to correspond to a pre-determined setpoint temperature of the user,
wherein said controller is configured for communicating said pre-determined setpoint temperature to the water heater for controlling the supply of water to said pre-determined setpoint temperature.

2. The shower head of claim 1, wherein said controller is configured to automatically change said pre-determined setpoint temperature upon a duration of use of said shower head to increase the comfort of the user.

3. The shower head of claim 1, wherein said controller is configured to automatically increase said pre-determined setpoint temperature upon a duration of use of said shower head to increase the comfort of the user.

4. The shower head of claim 3, wherein said controller further comprises a high temperature limit to which said increase is limited.
